(19) United States
(12) Patent Application Publication
(10) Pub. No.: US 2020/0078381 A1
(43) Pub. Date: Mar. 12, 2020

(54) LIPOSOMAL ENCAPSULATION METHOD AND DEVICES

(71) Applicants: Brett Delinski, Las Vegas, NV (US); Albert Mika, Las Vegas, NV (US)

(72) Inventors: Brett Delinski, Las Vegas, NV (US); Albert Mika, Las Vegas, NV (US)

(21) Appl. No.: 16/126,781

(22) Filed: Sep. 10, 2018

(51) Int. Cl. A61K 31/685; A61K 31/352; A61K 9/107; A61K 9/127; A61K 9/28; A61K 9/48

(52) U.S. Cl. CPC A61K 31/685; A61K 31/352; A61K 9/4808; A61K 9/1272; A61K 9/282; A61K 9/107

(57) ABSTRACT

The embodiments disclose a method including using a sonication process to encapsulate a human consumable substance within a liposome vesicle, continuously cooling the liposome vesicle and the encapsulated human consumable substance to a predetermined temperature during the sonication process, adding starch and essential oils to the cooled liposome vesicle and the encapsulated human consumable substance to create a mixture, using at least one analytical test for checking predetermined quality control standards of the encapsulated human consumable ingredients, mixtures and finished products, and using at least one delivery form for the mixture suitable human consumption.

### BACKGROUND

The consumer use of over-the-counter supplements, medications, and other health remedies has increased. Many of the over-the-counter products including a vast array of new products do not meet the expectation of the consumer in terms of bioavailability and absorption. Consumers want full benefits from supplements, medications, and other health remedies and quickly acting relief.

### BRIEF DESCRIPTION OF THE DRAWINGS

FIG. 1 shows a block diagram of an overview of a liposomal encapsulation method and devices of one embodiment.

FIG. 2 shows a block diagram of an overview of a flow chart of mixing a cannabinoid solution and a lipid solution of one embodiment.

FIG. 3A shows a block diagram of an overview of a flow chart of ultrasonic cell disruptor probe cell disruption of one embodiment.

FIG. 3B shows a block diagram of an overview of a flow chart of ultrasonic cell disruptor horn cup cell disruption of one embodiment.

FIG. 3C shows a block diagram of an overview of a flow chart of ultrasonic cell disruptor probe continuous cell disruption of one embodiment.

FIG. 3D shows a block diagram of an overview of a flow chart of a sonication bath cell disruption of one embodiment.

FIG. 4 shows a block diagram of an overview of a flow chart of a liposomal encapsulated cannabinoids starch coating of one embodiment.

FIG. 5 shows a block diagram of an overview of product vehicle packaging of one embodiment.

FIG. 6A shows for illustrative purposes only an example of small cavitation bubbles of one embodiment.

FIG. 6B shows for illustrative purposes only an example of enlarged cavitation bubbles of one embodiment.

FIG. 6C shows for illustrative purposes only an example of cavitation bubble implosion of one embodiment.

FIG. 6D shows for illustrative purposes only an example of cannabinoid compound disruption from cavitation bubble implosions of one embodiment.

FIG. 7A shows for illustrative purposes only an example of a lipid molecule of one embodiment.

FIG. 7B shows for illustrative purposes only an example of a phospholipid bilayer of one embodiment.

FIG. 7C shows for illustrative purposes only an example of a liposome vesicle of one embodiment.

FIG. 7D shows for illustrative purposes only an example of a liposomal encapsulated material of one embodiment.

FIG. 8 shows for illustrative purposes only an example of a liposomal encapsulated material transfer to a human cell of one embodiment.

FIG. 9A shows for illustrative purposes only an example of ultrasonic cell disruptor probe sonication of one embodiment.

FIG. 9B shows for illustrative purposes only an example of ultrasonic bath sonication of one embodiment.

FIG. 10 shows for illustrative purposes only an example of an ultrasonic cell disruptor horn cup sonication process of one embodiment.

FIG. 11 shows for illustrative purposes only an example of an ultrasonic cell disruptor probe continuous robotic sonication process of one embodiment.

FIG. 12 shows for illustrative purposes only an example of encapsulation processing apparatus of one embodiment.

FIG. 13 shows for illustrative purposes only an example of encapsulated cannabinoid product processing apparatus of one embodiment.

FIG. 14 shows for illustrative purposes only an example of liposomal encapsulated products of one embodiment.

FIG. 15A shows for illustrative purposes only an example of a continuous ultrasonic cell disruptor bath process of one embodiment.

FIG. 15B shows for illustrative purposes only an example of a continuous ultrasonic cell disruptor bath process high frequency pattern of one embodiment.

FIG. 16A shows for illustrative purposes only examples of liposomal products use of one embodiment.

FIG. 16B shows for illustrative purposes only a continuation of examples of liposomal products use of one embodiment.

FIG. 17A shows for illustrative purposes only examples of cannabinoid components liposomal product uses of one embodiment.

FIG. 17B shows for illustrative purposes only examples of CBD component liposomal product use of one embodiment.

FIG. 18 shows a block diagram of an overview of a liposomal encapsulation process testing of one embodiment.

### DETAILED DESCRIPTION OF THE INVENTION

In a following description, reference is made to the accompanying drawings, which form a part hereof, and in which is shown by way of illustration a specific example in which the invention may be practiced. It is to be understood that other embodiments may be utilized and structural changes may be made without departing from the scope of the embodiments.

### General Overview

It should be noted that the descriptions that follow, for example, in terms of liposomal encapsulation method and devices is described for illustrative purposes and the underlying system can apply to any number and multiple types of supplements, medications, and other health remedies. In one embodiment of the present invention, the liposomal encapsulation method and devices includes human consumable substances including vitamins, minerals, cannabinoids, medicinal substances and compounds and other legal products. The liposomal encapsulation method and devices includes legal cannabidiol (CBD) oil distillate and includes vitamins, minerals and other supplements and medications using the embodiments.

In one embodiment human consumables can include legalized and medicinal cannabis products including legal medicinal cannabis tobacco products, cannabidiol antioxidant drugs, cannabinoids not including THC, HU-210, HU-211 or any other NMDA receptor antagonist and wherein the cannabinoid is not psychoactive, and is not psychotoxic even at high doses, and legal medicinal cannabis patient prescription medications.

In another embodiment human consumables can include vitamins including for example vitamins C, D, B12, and others, minerals including for example calcium, zinc, iron and others, electrolytes, over the counter remedies for example antihistamines, and prescription medications following FDA and local regulations. In yet another embodiment human consumables can include can include food components and nutrients for example proteins, carbohydrates, high-density lipoprotein (HDL) cholesterol and others.

Human consumables delivered using the embodiments are health aids to the user for supplemental aids for nutritional deficiencies, long term non-toxic use to overcome physiological inabilities to properly absorb needed nutritional components. In addition, human consumables can include long term non-addictive pain management medications and formulations to, for example, replace and/or reduce opioid and other similar addictive medications for treating conditions normally treated with addictive pain medications. The embodiments are available in various delivery forms to accommodate users who for example may be averse to needles, have difficulty swallowing pills and tablets, where the user may not adequately take the human consumable due to these aversions or difficulties.

For example adding the liposomal encapsulated human consumable to foods make it possible for a user to gain the benefits while simply eating a bowl of soup, dried fruits and vegetables, eating baked goods where the liposomal encapsulated human consumable has been added to baking flour, using a suppository, applying a liposomal encapsulated human consumable cream to a tampon for a vaginal application to absorb the human consumable of one embodiment.

The drawings and descriptions are using cannabinoid processing as an example of the method and types of products that gain bioavailability and absorption improvements from the processes disclosed. For example the Increase of bioavailability and the absorption of the cannabinoids from the standard 4-12% increase up to 90% through the process of liposomal encapsulation of cannabinoids in a lipid solution. Through this process it also increases the uptake so supplements, medications, and other health remedies start to work within 15-30 minutes versus a standard 60-120 minutes.

In this example the legality of the distribution, sale of cannabinoid compounds is changing quickly and in diverse manners. The processing and product production of cannabinoid compounds will be in accordance with all applicable federal, state and local laws.

In the embodiments the encapsulation of human consumable substances takes place in an aqueous or oil solution and/or emulsion depending on the nature of the human consumable material and lipid material.

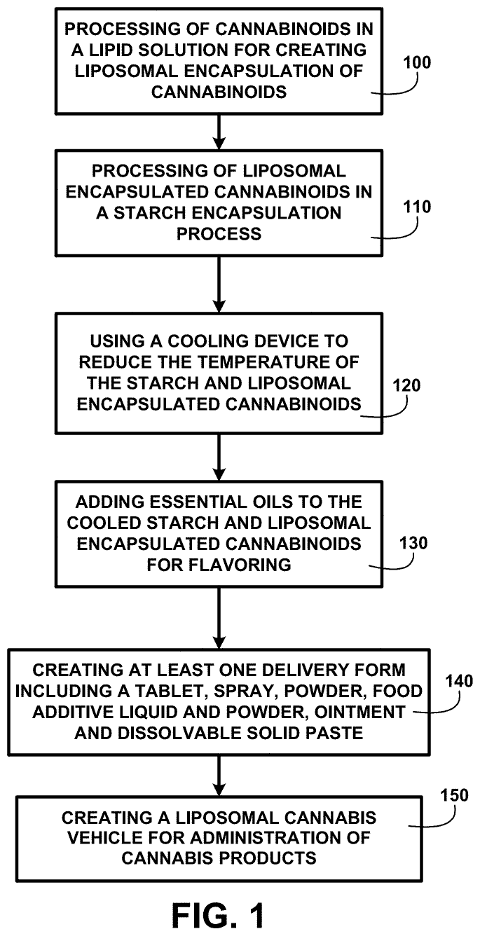

FIG. 1 shows a block diagram of an overview of a liposomal encapsulation method and devices of one embodiment. FIG. 1 shows a method for processing of cannabinoid distillate in a lipid solution for creating liposomal encapsulation of cannabinoids 100. Processing of liposomal encapsulated cannabinoids in a starch encapsulation process 110 for extending a shelf life of final products. Liposomal encapsulation sonication adds heat to the solution due to implosive energy released by ultrasonic cavitation bubbles. The processing includes using a cooling device to reduce the temperature of the starch and liposomal encapsulated cannabinoids 120. Adding essential oils to the cooled starch and liposomal encapsulated cannabinoids for flavoring 130 the liposomal encapsulated cannabinoids.

Creating at least one delivery form including a tablet, spray, powder, food additive liquid and powder, ointment and dissolvable solid paste 140. Creating a liposomal cannabis vehicle for administration of cannabis products 150. Using essential oils for flavoring also applies to starch and liposomal encapsulated products made with for example vitamins and minerals and delivery forms including food additives, bottled and powdered drinks, dried fruits and vegetable, freeze dried fruits and vegetables, plant juices, live powdered drink mixes, live plant powders and other groups of human consumable substances of one embodiment.

### DETAILED DESCRIPTION

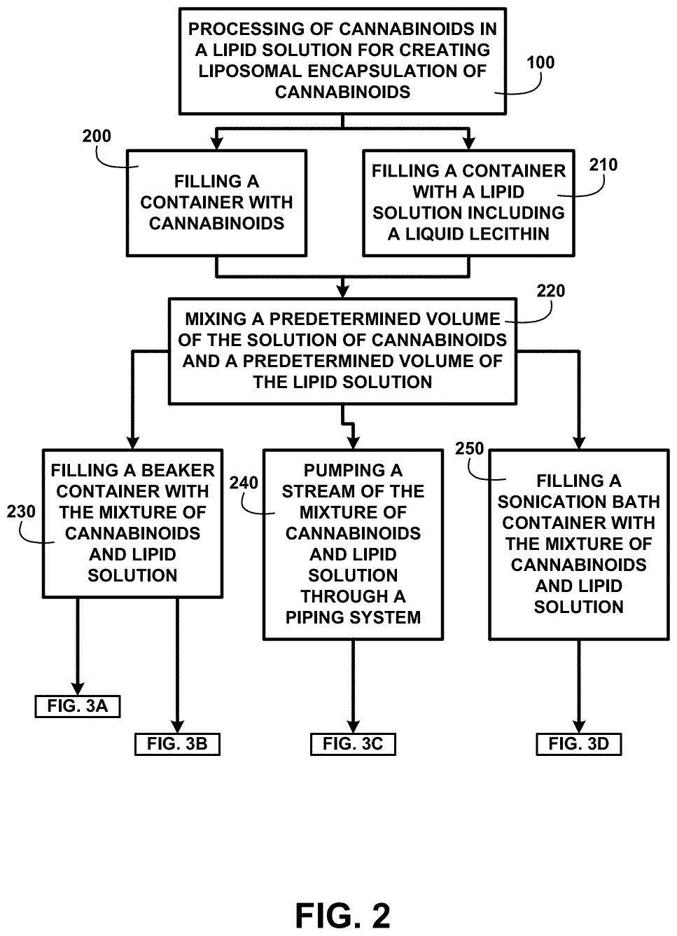

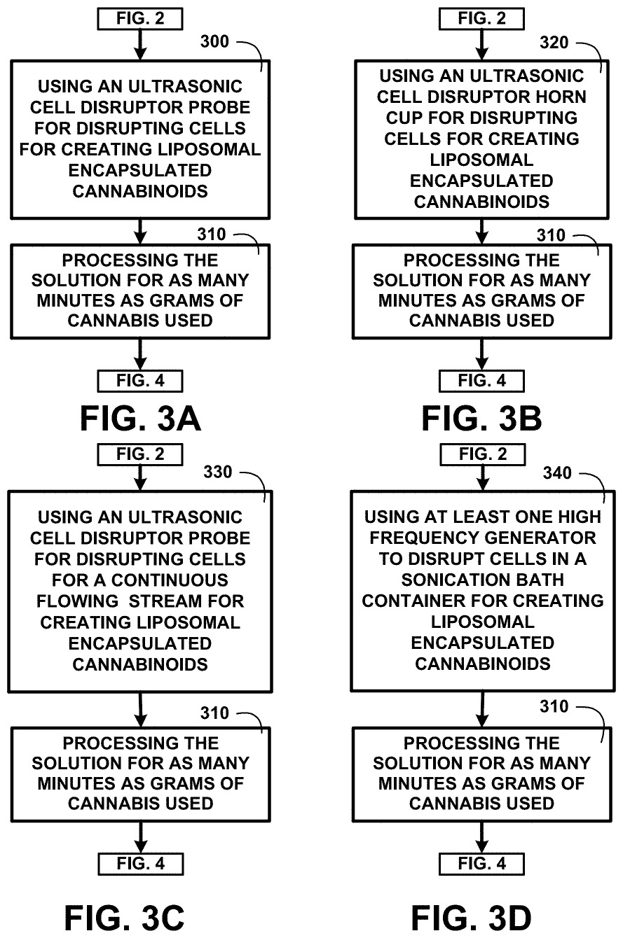

FIG. 2 shows a block diagram of an overview of a flow chart of mixing a cannabinoid solution and a lipid solution of one embodiment. FIG. 2 shows processing of cannabinoids in a lipid solution for creating liposomal encapsulation of cannabinoids 100. Filling a container with cannabinoids 200 and filling a container with a lipid solution including a liquid lecithin 210. The filling process of the liquid lecithin 210 solution plus a human consumable material uses a one to one ratio plus a range from 7 to 10 extra grams of lecithin depending on a product application.

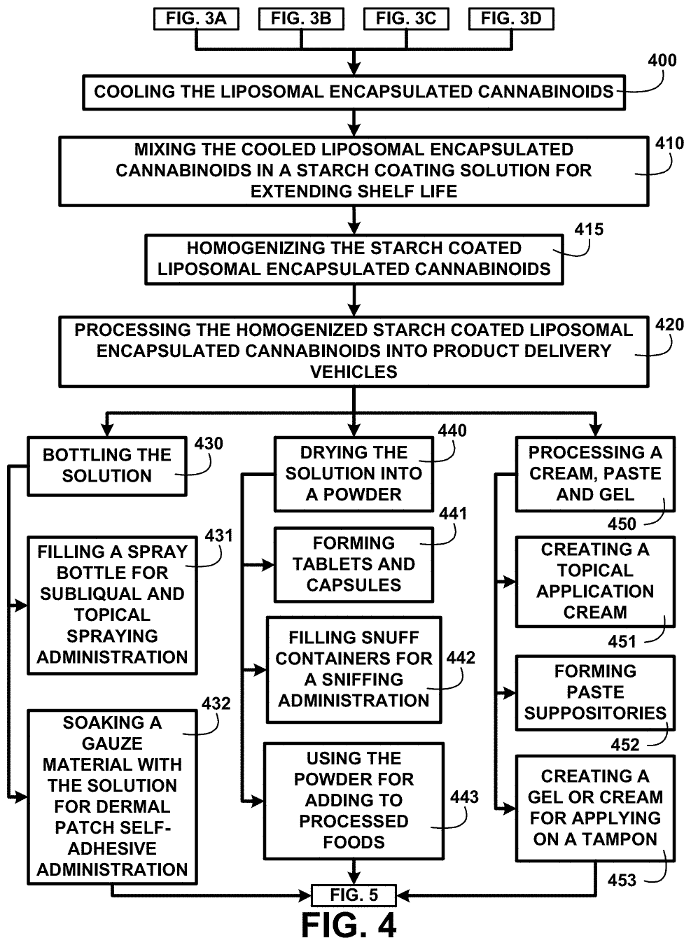

Using the substances filled in the containers the process begins mixing a predetermined volume of the solution of cannabinoids and a predetermined volume of the lipid solution 220 then filling a beaker container with the mixture of cannabinoids and lipid solution 230. This process continues in FIG. 3A and FIG. 3B. Pumping a stream of the mixture of cannabinoids and lipid solution through a piping system 240 begins a continuous processing of the mixture. This process continues in FIG. 3C. Another process begins with filling a sonication bath container with the mixture of cannabinoids and lipid solution 250. This process continues in FIG. 3D.

### Ultrasonic Cell Disruptor Probe Cell Disruption:

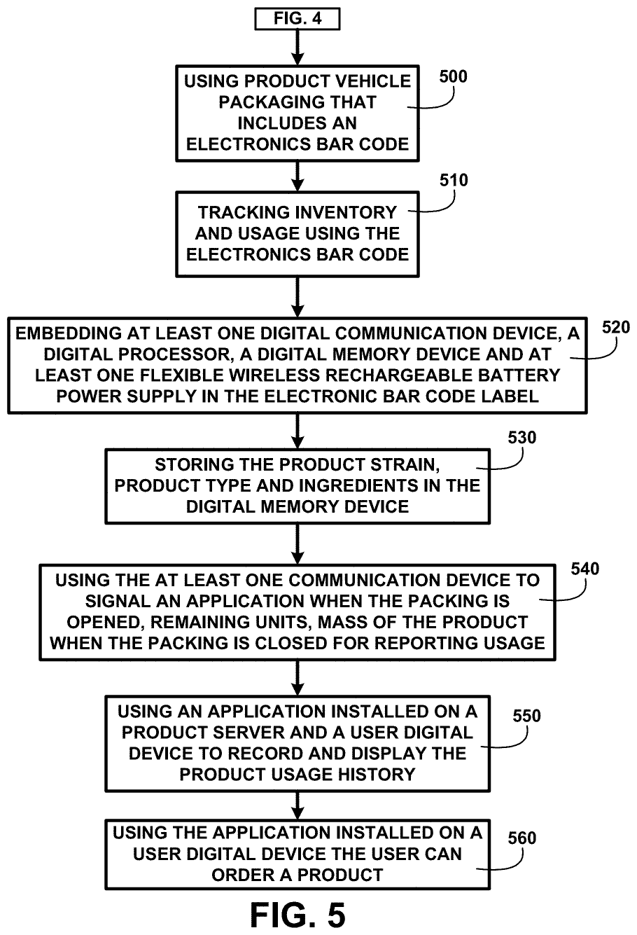

FIG. 3A shows a block diagram of an overview of a flow chart of ultrasonic cell disruptor probe cell disruption of one embodiment. FIG. 3A shows the processing continued from FIG. 2 with using an ultrasonic cell disruptor probe for disrupting cells for creating liposomal encapsulated cannabinoids 300 and processing the solution for as many minutes as grams of cannabis used 310. This process continues in FIG. 4.

### Ultrasonic Cell Disruptor Horn Cup Cell Disruption:

FIG. 3B shows a block diagram of an overview of a flow chart of ultrasonic cell disruptor horn cup cell disruption of one embodiment. FIG. 3B shows the process continuing from FIG. 2 including using an ultrasonic cell disruptor horn cup for disrupting cells for creating liposomal encapsulated cannabinoids 320. Processing the solution for as many minutes as grams of cannabis used 310 of one embodiment. This process continues in FIG. 4.

### Ultrasonic Cell Disruptor Probe Continuous Cell Disruption:

FIG. 3C shows a block diagram of an overview of a flow chart of ultrasonic cell disruptor probe continuous cell disruption of one embodiment. FIG. 3C shows continuing from FIG. 2 a process using an ultrasonic cell disruptor probe for disrupting cells a continuous flowing stream for creating liposomal encapsulated cannabinoids 330. Processing the solution for as many minutes as grams of cannabis used 310 of one embodiment. This process continues in FIG. 4

### A Sonication Bath Cell Disruption:

FIG. 3D shows a block diagram of an overview of a flow chart of a sonication bath cell disruption of one embodiment. FIG. 3D shows a continuance of the process descriptions from FIG. 2. Using at least one high frequency generator to disrupt cells in a sonication bath container for creating liposomal encapsulated cannabinoids 340. Processing the solution for as many minutes as grams of cannabis used 310 of one embodiment. This process continues in FIG. 4.

### A Liposomal Encapsulated Cannabinoids Starch Coating:

FIG. 4 shows a block diagram of an overview of a flow chart of a liposomal encapsulated cannabinoids starch coating of one embodiment. FIG. 4 shows a continuation from FIG. 3A, FIG. 3B, FIG. 3C AND FIG. 3D including cooling the liposomal encapsulated cannabinoids 400 after the encapsulation process. Mixing the cooled liposomal encapsulated cannabinoids in a starch coating solution for extending shelf life 410. The starch coating includes starches including maltodextrin. Homogenizing the starch coated liposomal encapsulated cannabinoids 415.

Processing the homogenized starch coated liposomal encapsulated cannabinoids into product delivery vehicles 420. The added bioavailability and rapid absorption created using liposomal encapsulated products speeds relief in various liquid forms including bottling the solution 430 for drinking, filling a spray bottle for sublingual and topical spraying administration 431, soaking a gauze material with the solution for dermal patch delivery self-adhesive administration 432 of one embodiment.

The added bioavailability and rapid absorption created using liposomal encapsulated products speeds relief in various powder forms including drying the solution into a powder 440 for forming tablets and capsules 441, filling snuff containers for a sniffing administration 442, and using the powder for adding to processed foods 443 ingredients. Processing a cream, paste and gel 450 also provides a convenient quick relief gained by the added bioavailability and rapid absorption. Using liposomal encapsulated products speeds relief with for example creating a topical application cream 451.

One group of liposomal encapsulated products includes forming paste suppositories 452 for digestive tract condition treatments including constipation, diverticulosis, Crohn's disease and other digestive tract conditions. Another form of liposomal encapsulated products include creating a gel or cream for applying on a tampon 453 for delivery of a liposomal encapsulated product for example a PMS medication, an endometriosis treatment medication and other female related conditions of one embodiment. The processing is further described in FIG. 5.

### Product Vehicle Packaging:

FIG. 5 shows a block diagram of an overview of product vehicle packaging of one embodiment. FIG. 5 shows a continuation from FIG. 4 including using product vehicle packaging that includes an electronic barcode 500. Tracking inventory and usage using the electronic barcode 510 includes embedding at least one digital communication device, a digital processor, a digital memory device and at least one flexible wireless rechargeable battery power supply in the electronic barcode label 520. Storing the product strain, product type and ingredients in the digital memory device 530 provides the data for inventory purposes and identifies the product being used.

Tracking usage is accomplished using the at least one communication device to signal an application when the packing is opened, remaining units, mass of the product when the packing is closed for reporting usage 540. The tracking usage data is accessed using an application installed on a product server and a user digital device for recording and displaying the product usage history 550. The digital processor is used for alerting a user when the remaining units and mass of the product falls below an adjustable threshold quantity. Upon receiving the alert on the user digital device the user using the application installed on a user digital device the user orders a product 560 of one embodiment.

### Small Cavitation Bubbles:

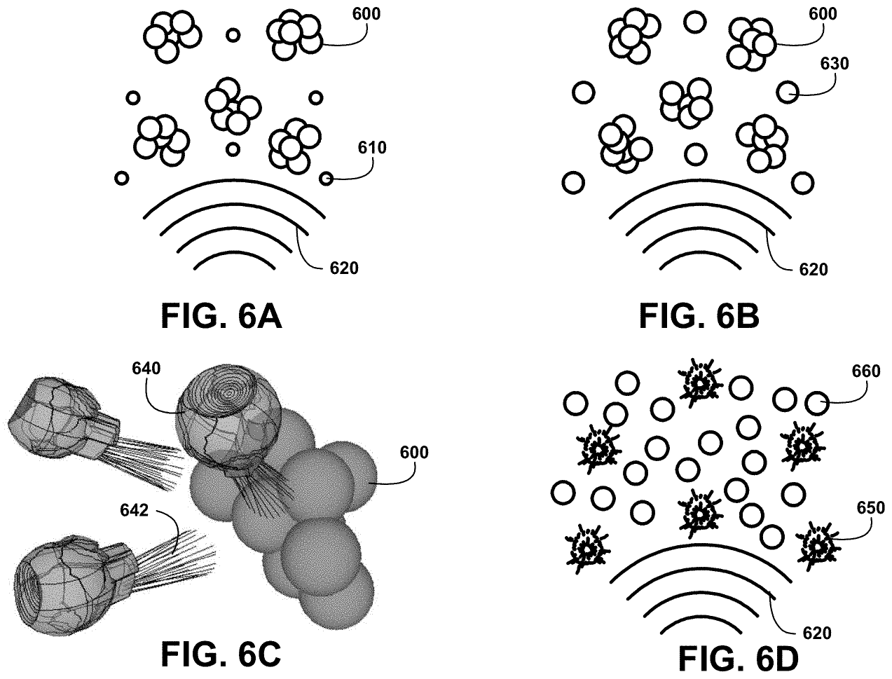

FIG. 6A shows for illustrative purposes only an example of small cavitation bubbles of one embodiment. FIG. 6A shows CBD compound molecules 600. The CBD compound molecules 600 are surrounded by cavitation small bubbles 610 created by sonication high frequency waves 620. This type of cavitation is generally referred to as acoustic cavitation of one embodiment.

### Enlarged Cavitation Bubbles:

FIG. 6B shows for illustrative purposes only an example of enlarged cavitation bubbles of one embodiment. FIG. 6B shows the CBD compound molecules 600 and sonication high frequency waves 620. Cavitation bubbles are enlarged 630 over time as more sonication high frequency energy is absorbed by the bubbles of one embodiment.

### Cavitation Bubble Implosion:

FIG. 6C shows for illustrative purposes only an example of cavitation bubble implosion of one embodiment. FIG. 6C shows the CBD compound molecules 600 and enlarged cavitation bubbles starting to implode 640. The imploding bubbles begin releasing energy stored from the ultrasound cavitation energy absorbed when the bubble forms and increases as the bubble is enlarged. The energy release is high speed and directional 642.

The dynamics of a single spherically oscillating bubble is well understood. However, when there is a nearby surface, the bubble often collapses non-spherically with a high-speed jet. The direction of the jet depends on the ‘resistance’ of the boundary: the bubble jets towards a rigid boundary, splits up near an elastic boundary, and jets away from a free surface. FIG. 6C shows the nearby CBD compound molecules 600 which form a rigid boundary. The high-speed jet of energy released by the implosion is directed towards the CBD compound molecules 600 rigid boundary of one embodiment.

Cannabinoid Compound Disruption from Cavitation Bubble Implosions:

FIG. 6D shows for illustrative purposes only an example of cannabinoid compound disruption from cavitation bubble implosions of one embodiment. FIG. 6D shows the sonication high frequency waves 620 creating and enlarging the cavitation bubbles. The enlargement of the cavitation bubbles ends when the cavitation bubbles implode. FIG. 6D shows enlarged cavitation bubbles imploded 650 which release the high-speed jet of energy. The high-speed jet of energy from one or more enlarged cavitation bubble causes the CBD compound molecules to separate into individual molecules 660 of one embodiment.

### A Lipid Molecule:

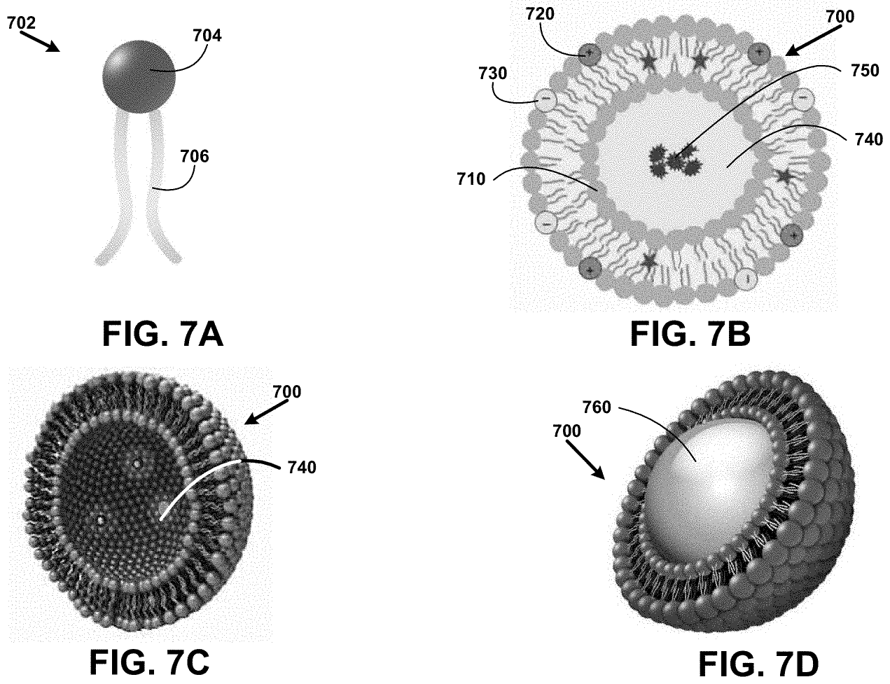

FIG. 7A shows for illustrative purposes only an example of a lipid molecule of one embodiment. FIG. 7A shows a lipid molecule 702 consisting of a hydrophilic head 704 and a hydrophobic tail 706. Liposomes are created when a high quality phospholipid such as phosphatidylcholine, a molecule derived from lecithin, is placed in water and consequently forms a series of bilayers each separated by water. However, when the lipids form micelles the hydrophobic tails interact with each other rather than with water and the formation of liposomes in a non-aqueous solution takes place. When membrane phospholipids are disrupted, they reassemble themselves into tiny spheres, smaller than a normal cell, either as bilayers or monolayers.

The bilayer structures are liposomes. The monolayer structures are called micelles. Lipid spheres that contain no aqueous material are called micelles. Human consumable substances with an aqueous nature are encapsulated in lecithin liposomes and human consumable substances with a non-aqueous nature are encapsulated in lecithin micelles.

Processing liquid lecithin plus for example a cannabis extract including CBD distillate in a one to one ratio plus 7 to 10 extra grams of lecithin depending on the application it is used in for proper encapsulation in the final end product. In the liposomal encapsulation method and devices the preferred liquid lecithin is an organic non-GMO Sunflower oil which includes an elevated hypoallergenic characteristic. Another liquid lecithin includes organic and non-organic soy oil. The mixed solution is processed using sonication that includes using an ultrasonic cell disruptor including a probe, horn cup and/or bath for as many minutes as grams of cannabis or other human consumable product material. The encapsulation is achieved once enough energy has been supplied under the sonication process of one embodiment.

### A Phospholipid Bilayer:

FIG. 7B shows for illustrative purposes only an example of a phospholipid bilayer of one embodiment. FIG. 7B shows a phospholipid bilayer 700 composed of a hydrophilic head with a positive charge 720, a hydrophilic head with a negative charge 730 and the respective hydrophobic tail 706 of FIG. 7A. Phospholipids including phosphatidylcholine are amphiphilic; they consist of a hydrophilic (water loving) head and hydrophobic (water hating) tail. When phospholipids are placed in an aqueous solution, the hydrophobic tails face each other avoiding the water and form a phospholipid bilayer, while the hydrophilic heads form hydrogen bonds with the water molecules.

The lipid bilayer will form a closed sphere (liposome) to completely exclude water from the hydrophobic tail. The formation of the liposome is seen wherein hydrophilic heads form in an opposing hydrophobic position 710 due to the hydrophobic tail 706 of FIG. 7A avoidance of water. The interior of the liposome forms a spherical vesicle 740 in which encapsulated material 750 is protected. A liposome is a spherical vesicle having at least one lipid bilayer. The liposome is used as a vehicle for administration of nutrients and pharmaceutical drugs of one embodiment.

### A Liposome Vesicle:

FIG. 7C shows for illustrative purposes only an example of a liposome vesicle of one embodiment. FIG. 7C shows the phospholipid bilayer 700 which forms the liposome spherical vesicle 740 of one embodiment.

### A Liposomal Encapsulated Material:

FIG. 7D shows for illustrative purposes only an example of a liposomal encapsulated material of one embodiment. FIG. 7D shows the phospholipid bilayer 700 and substance encapsulated in the phospholipid bilayer liposome spherical vesicle with a liposome encapsulated substance 760 including liposomal encapsulation method and devices product substances of one embodiment.

### A Liposomal Encapsulated Material Transfer to a Human Cell:

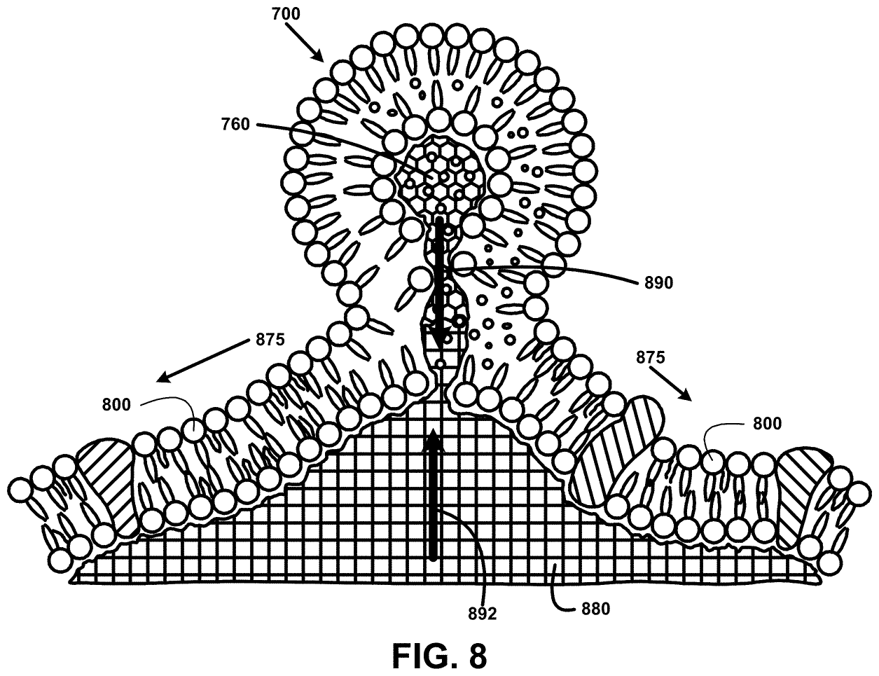

FIG. 8 shows for illustrative purposes only an example of a liposomal encapsulated material transfer to a human cell of one embodiment. FIG. 8 shows the phospholipid bilayer 700 of FIG. 7A with the liposome encapsulated substance 760 of FIG. 7D. A human cell membrane 800 is composed of two layers of lipid molecules a lipid bilayer. The human cell membrane 800 lipid molecules each have a hydrophilic and a hydrophobic end. Rapid absorption occurs with liposomal phospholipid bilayer spreading over a human cell 875.

The phospholipid bilayer 700 of FIG. 7A with the liposome encapsulated material 760 of FIG. 7D reacts with the human cell membrane 800 lipid bilayer wherein the hydrophobic characteristic of the hydrophobic tail 706 of FIG. 7A grouping creates a breach in the human cell membrane 800. Liposome encapsulated material passes through the breach into human cell 890 and human cell material 880 passes through the breach into an opened liposome vesicle 892 filling the void left by the passing encapsulated material thereby accelerating the rate of the transfer of substances. Once the transfers of the substances are completed the phospholipid bilayer 700 of FIG. 7A collapses across the surface of the human cell membrane 800 resealing the human cell membrane 800 and is absorbed through normal bodily functions of one embodiment.

### Ultrasonic Cell Disruptor Probe Sonication:

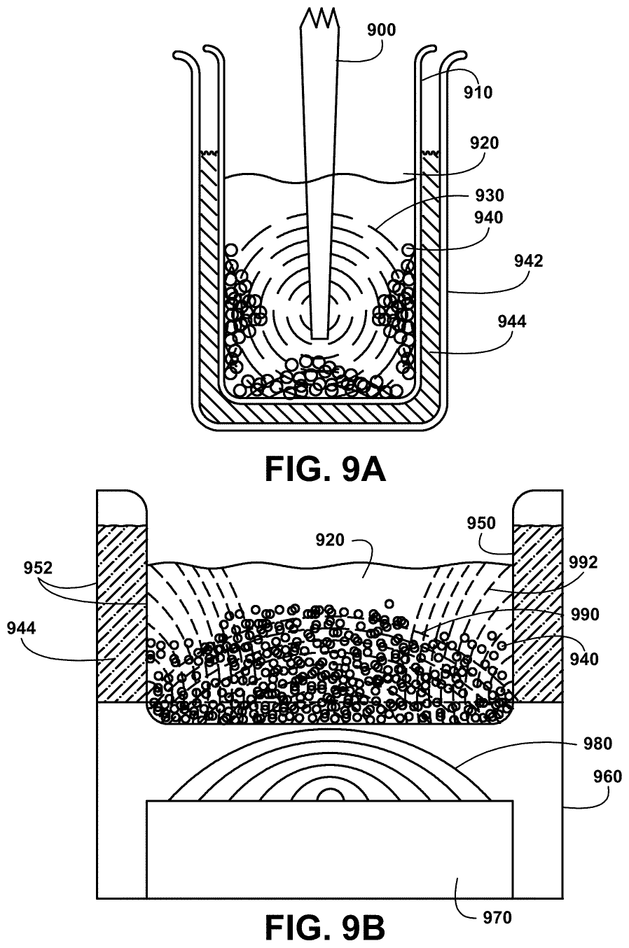

FIG. 9A shows for illustrative purposes only an example of ultrasonic cell disruptor probe sonication of one embodiment. FIG. 9A shows an ultrasonic cell disruptor probe 900 in an aqueous solution held in a beaker 910. A solution 920 is directly impacted by ultrasonic probe direct sonication high frequency waves 930. The sonication high frequency wave energy creates cavitation bubbles 940. The sonication process increases the temperature of the aqueous solution held in the beaker. Temperatures above 220 degrees C. during processing causes degradation of the aqueous solution substances.

The liposomal encapsulation method employs to processes and devices to maintain a temperature below 220 degrees C. A first temperature control method is to cycle the ultrasonic cell disruptor probe 900 operation. The ultrasonic cell disruptor probe 900 operation is cycled with 30 seconds on followed by 2 minutes off in a repeating cycle pattern. A second temperature control method is to place the beaker 910 in a chilling vessel 942 that contains a cooling media 944. The cooling media 944 circulates through piping to and from a cooling apparatus (not shown). The cooling media 944 experiences a heat transfer from the increased temperature aqueous solution. This reduces the temperature of the aqueous solution to a predetermined temperature.

The cooling media 944 increases in temperature. The cooling apparatus (not shown) removes heat from the cooling media 944 and conveys it back to the chilling vessel 942 to repeat this continuous process of one embodiment. The amplitude of frequency of the ultrasonic probe direct sonication high frequency waves 930 is adjusted. The liposomal encapsulation method and devices automatically adjust the amplitude of frequency being applied in a range from 2-70 kHz depending on the specific machine being used and volume of the aqueous solution of one embodiment.

### Ultrasonic Bath Sonication:

FIG. 9B shows for illustrative purposes only an example of ultrasonic bath sonication of one embodiment. FIG. 9B shows a sonication bath container 950 coupled to a sonication bath device 960. The sonication bath container 950 holds the aqueous solution 920. The sonication bath container 950 includes a chilling vessel surround 952 along the perimeter of the bath container. The chilling vessel surround 952 holds the cooling media 944 that circulates between the cooling apparatus (not shown) in a heat transfer process to control the temperature of the aqueous solution 920.

The sonication bath device 960 includes at least one high frequency generator 970. Ultrasonic high frequency generator directed sonication high frequency waves 980 directly impact the sonication bath container 950. The energy impacted on the sonication bath container 950 floor in this example causes indirect high frequency waves on the interior of the floor material and creates high frequency generator indirect sonication bath high frequency waves 990 in the aqueous solution 920. The walls of the sonication bath device 960 reflect sonication bath high frequency waves shown without material in the solution for clarity 992. Cavitation bubbles 940 form on the floor and walls of one embodiment.

### An Ultrasonic Cell Disruptor Horn Cup Sonication Process:

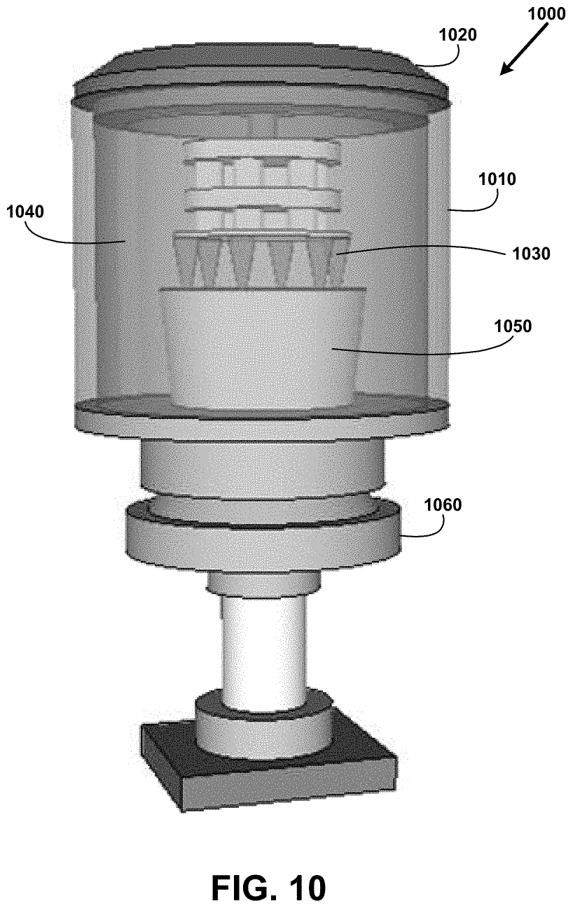

FIG. 10 shows for illustrative purposes only an example of an ultrasonic cell disruptor horn cup sonication process of one embodiment. FIG. 10 shows an ultrasonic cell disruptor horn cup sonication process 1000 using an ultrasonic cell disruptor horn cup 1020 and a horn cup container with water 1010. Water 1040 in the container receives direct sonication high frequency waves. The impact of the high frequency waves generated using an ultrasonic cell disruptor horn cup activator 1060 to oscillate an ultrasonic cell disruptor horn 1050 through the water to cannabinoid and lipid solution in vials 1030 cause indirect sonication high frequency waves within the vials. The indirect sonication high frequency waves create cavitation within the vials and formation of cavitation bubbles. The cavitation bubbles implode in the vials and create encapsulated cannabinoid solution of one embodiment.

### Ultrasonic Cell Disruptor Probe Continuous Robotic Sonication Process:

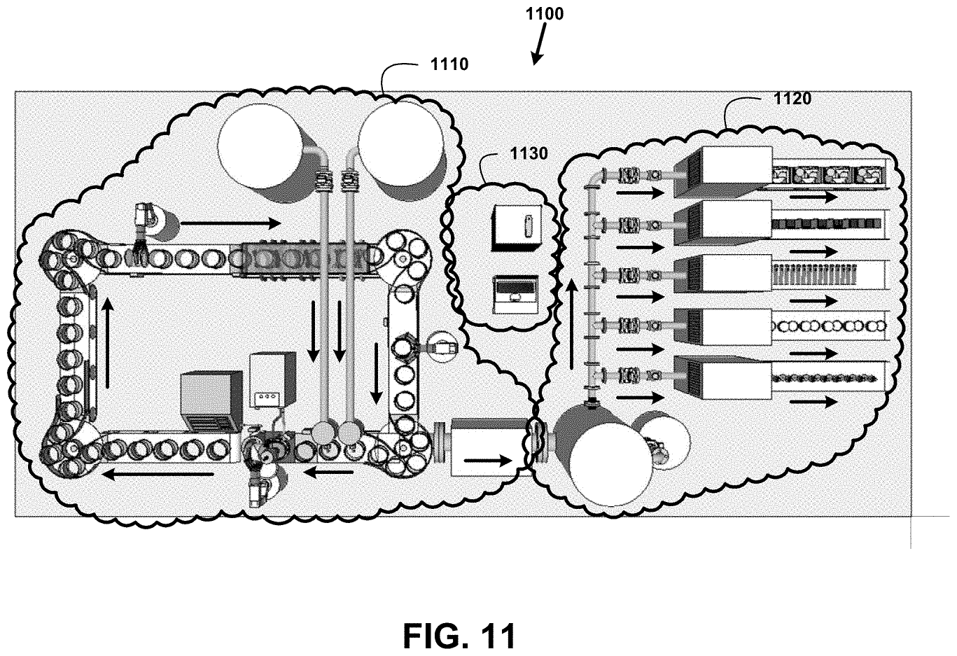

FIG. 11 shows for illustrative purposes only an example of an ultrasonic cell disruptor probe continuous robotic sonication process of one embodiment. FIG. 11 shows an ultrasonic cell disruptor probe continuous robotic sonication process 1100 including an ultrasonic cell disruptor probe continuous robotic sonication process apparatus 1110, an encapsulated cannabinoid product processing apparatus 1120, and a continuous robotic sonication process and product process control apparatus 1130 of one embodiment.

### Encapsulation Processing Apparatus:

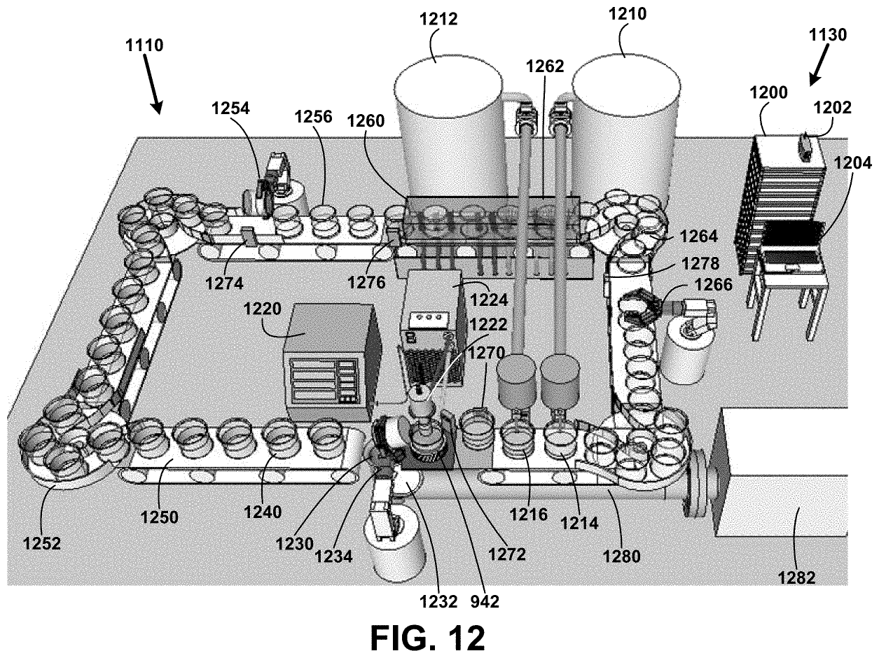

FIG. 12 shows for illustrative purposes only an example of encapsulation processing apparatus of one embodiment. FIG. 12 shows the ultrasonic cell disruptor probe continuous robotic sonication process apparatus 1110 and continuous robotic sonication process and product process control apparatus 1130. The continuous robotic sonication process and product process control apparatus 1130 includes a sonication process and product process control digital server 1200, a Wi-Fi communication device 1202 and at least one computer 1204.

The sonication process and product process control digital server 1200 tracks, regulates and controls progress of processing segments using data received from at least one wireless sensor. At least one wireless optical level sensor 1270 is used for measuring the volume of cannabinoid supply deposited into a beaker prior to sonication. The volume of cannabinoid supply is predetermined to adjust to a beaker size and is entered using the at least one computer 1204 by user. The predetermined volume quantity is stored on a database in the sonication process and product process control digital server 1200. The at least one wireless optical level sensor 1270 transmits a WI-FI volume signal to the sonication process and product process control digital server 1200 using the WI-FI communication device 1202. When a predetermined volume of cannabinoid has been deposited into a beaker 1214 the sonication process and product process control digital server 1200 closes a supply valve stopping the flow of cannabinoid from a cannabinoid supply tank 1210 of one embodiment.

At least one wireless optical level sensor 1270 is used for measuring the volume of lipid solution supply from a lipid solution supply tank 1212 deposited into a beaker with a predetermined cannabinoid volume 1216 prior to sonication. The lipid solution supply valve is closes by a signal from the sonication process and product process control digital server 1200 upon receiving a wireless optical level sensor 1270 transmitted WI-FI signal that a predetermined volume of lipid solution has been deposited of one embodiment.

Once both the cannabinoid and lipid solution have been deposited into a beaker the sonication process and product process control digital server 1200 transmits a signal to a robotic gripper 1234 to place the filled beaker into a chilling vessel 942. The chilling vessel 942 holds in an interior chamber a cooling media 944 of FIG. 9A that circulates from a cooling apparatus 1224. When the beaker is placed in the chilling vessel 942 a pressure sensor not shown signals the sonication process and product process control digital server 1200 that a beaker is in place for sonication. The sonication process and product process control digital server 1200 transmits a signal to an ultrasonic cell disruptor probe apparatus 1220. The ultrasonic cell disruptor probe apparatus 1220 lowers an ultrasonic cell disruptor probe 1222 a predetermined distance into the beaker and begins a sonication process of one embodiment.

An interior temperature of the mixture of cannabinoid and lipid solution increases with the vibrations of the ultrasonic cell disruptor probe 1222 and the temperature of the tip of the ultrasonic cell disruptor probe 1222 also increases in temperature. This generally causes a stoppage in the sonication process to prevent overheating and damage to the probe and solution. Circulating a chilled media through the chilling vessel 942 continuously performs a heat transfer process to control the solution and probe tip temperature, prevent overheating and stoppage of the sonication process reducing the sonication process time. A wireless non-contact temperature sensor 1272 continuously determines the sonication temperature and transmits a signal to the sonication process and product process control digital server 1200 which transmits signals to the cooling apparatus 1224 for regulating the cooling temperatures of the cooling apparatus 1224 to prevent overheating of one embodiment.

The ultrasonic cell disruptor probe apparatus 1220 signals to the sonication process and product process control digital server 1200 when a predetermined period of time and intensity for sonication has elapsed. The sonication process and product process control digital server 1200 validates the time and intensity and transmits a signal to the ultrasonic cell disruptor probe apparatus 1220 to raise the ultrasonic cell disruptor probe 1222 out of the beaker. The ultrasonic cell disruptor probe apparatus 1220 transmits a signal when the probe has completed being raised of one embodiment.

The sonication process and product process control digital server 1200 transmits a signal to the robotic gripper 1234 to lift the sonication encapsulated cannabinoid solution beaker from the chilling vessel 942. The sonication process and product process control digital server 1200 transmits a signal to a solenoid not shown to open an encapsulated cannabinoid collection piping opening retractable cover 1232 on the encapsulated cannabinoid collection piping opening 1230. The robotic gripper 1234 receives a signal transmitted from the sonication process and product process control digital server 1200 to position the beaker and pour the encapsulated cannabinoid solution into the encapsulated cannabinoid collection piping opening 1230 then place the emptied beaker 1240 onto a conveyor belt 1250. The poured encapsulated cannabinoid solution flows through encapsulated cannabinoid collection piping 1280 to an encapsulated cannabinoid homogenization apparatus 1282 where the encapsulated cannabinoid solution is homogenized for product processing of one embodiment.

A plurality of emptied beakers 1240 are then conveyed along the conveyor belt 1250 including a system of moving conveyor belt 1250 and conveyor belt carousel 1252 devices. A wireless beaker sensor 1274 detects an emptied beaker to activate an emptied beaker robotic gripper 1254 by transmitting a signal to the sonication process and product process control digital server 1200. The sonication process and product process control digital server 1200 transmits a signal to the robotic gripper 1254 to lift an invert the emptied beaker onto the conveyer. The robotic gripper 1254 places an inverted emptied beaker 1256 on the conveyor for processing in a steam cleaning apparatus 1260 and compressed air clean beaker dryer 1262 of one embodiment.

A wireless steam cleaner cycle sensor and activator 1276 detects the inverted emptied beaker 1256 when it reaches the steam cleaning apparatus 1260 and transmits a signal to the sonication process and product process control digital server 1200. The sonication process and product process control digital server 1200 transmits a signal to the steam cleaning apparatus 1260 to begin spraying steam onto the surfaces of the inverted emptied beaker 1256 to wash away any material residue. The compressed air clean beaker dryer 1262 begins blowing filtered heated compressed air against the surfaces of the steam cleaned inverted emptied beaker 1256 for drying the surfaces. When the cleaned beaker 1264 travels down a conveyor belt 1250 a wireless beaker sensor 1278 transmits a signal to the sonication process and product process control digital server 1200. The sonication process and product process control digital server 1200 transmits a signal to activate a cleaned beaker robotic gripper 1266 for inverting the cleaned beaker 1264 for cycling to the start of the cannabinoid supply deposition process of one embodiment.

### Encapsulated Cannabinoid Product Processing Apparatus:

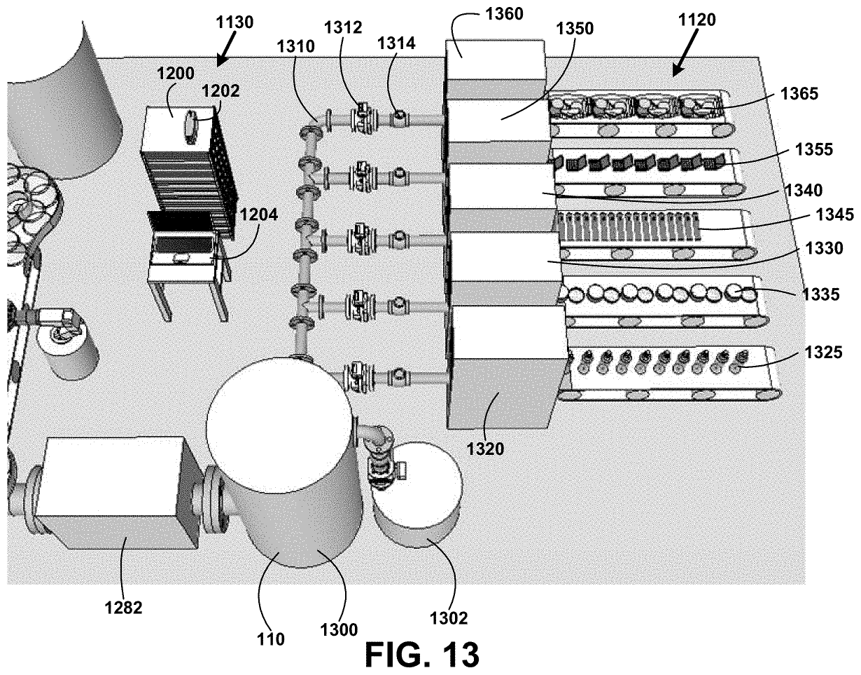

FIG. 13 shows for illustrative purposes only an example of an encapsulated cannabinoid product processing apparatus of one embodiment. FIG. 13 shows the encapsulated cannabinoid product processing apparatus 1120 that is monitored and regulated by the continuous robotic sonication process and product process control apparatus 1130. The poured encapsulated cannabinoid solution flows through encapsulated cannabinoid collection piping 1280 of FIG. 12 to an encapsulated cannabinoid homogenization apparatus 1282 where the encapsulated cannabinoid solution is homogenized for product processing with the encapsulated cannabinoid product processing apparatus 1120. The encapsulated cannabinoid product processing apparatus 1120 includes a supply of the homogenized encapsulated cannabinoid solution. The homogenized encapsulated cannabinoid solution is flowed through encapsulated cannabinoid product processing piping 1310.

The flow of the homogenized encapsulated cannabinoid solution is metered for volume using a wireless digital valve 1312 wherein the sonication process and product process control digital server 1200 transmits a WI-FI signal using the WI-FI communication device 1202. The volume of the homogenized encapsulated cannabinoid solution for each product type is entered using the at least one computer 1204 and stored in at least one database in the sonication process and product process control digital server 1200. The sonication process and product process control digital server 1200 transmitted signal opens and closes the wireless digital valve 1312 and the volume dispatched is metered through a wireless digital flow meter 1314. The dispatched volume data from the wireless digital flow meter 1314 is transmitted to the sonication process and product process control digital server 1200 for recording in product batch data.

The homogenized encapsulated cannabinoid solution from the homogenized encapsulated cannabinoid solution supply tank 1300 is further processed in the starch encapsulation process 110 of FIG. 1. A starch coating is supplied from a starch encapsulation process supply tank 1302. The starch coated homogenized encapsulated cannabinoid solution from the homogenized encapsulated cannabinoid solution is processed to create liposomal encapsulated products including for example liquid products for example a sublingual spray, dermal patch, soup and bottled drink, and powdered products for example baking flour, snuff, cream, gel, paste, and tablets.

The homogenized encapsulated cannabinoid solution is processed into ingredients for each type of product. FIG. 13 illustrates a liquid liposomal encapsulated product processor 1320 processing and creating a bottled sublingual spray 1325 for rapid absorption under a users' tongue to enjoy cannabinoid for rapid pain relief. Also illustrated is a topical cream liposomal encapsulated product processor 1330 showing the processing and creation of a topical cream liposomal encapsulated product 1335 wherein a user applies the topical cream and for example to gain rapid absorption of the liposomal encapsulated product including cannabinoid for pain relief.

A gel liposomal encapsulated product processor 1340 performs processing for creating a gel liposomal encapsulated product 1345 that delivers in one embodiment relieve conditions by applying a gel liposomal encapsulated product 1345 applied to a tampon. FIG. 13 illustrates a tablet liposomal encapsulated product processor 1350 for processing homogenized encapsulated cannabinoid solution into a powder form and compressing the powder for creating a tablet liposomal encapsulated product 1355.

A dried fruits and vegetables, freeze dried fruits and vegetables, plant juices, live powdered drink mixes, live plant powders liposomal encapsulated product processor 1360 for example marinates fruits and vegetable in the homogenized encapsulated cannabinoid solution prior to dehydration to create a packaged dried fruits and vegetables, freeze dried fruits and vegetables, plant juices, live powdered drink mixes, live plant powders liposomal encapsulated product 1365. The liposomal encapsulation method and devices produce homogenized encapsulated cannabinoid solution products for human consumption in one embodiment and homogenized encapsulated cannabinoid solution products for human use in another embodiment.

### Liposomal Encapsulated Products:

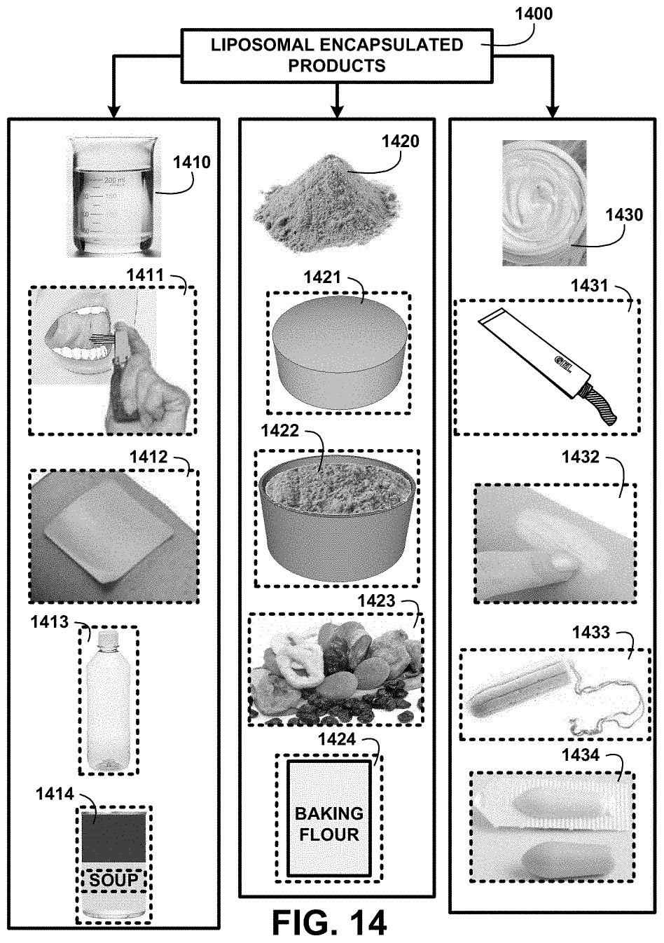

FIG. 14 shows for illustrative purposes only an example of liposomal encapsulated products of one embodiment. FIG. 14 shows liposomal encapsulated products 1400 including at least one liquid liposomal encapsulated product 1410 including for example a sublingual spray 1411, a dermal patch 1412, bottled drinks 1413 and liquid processed foods for example soup 1414 and plant juices. The liposomal encapsulated products 1400 include a powder liposomal encapsulated product 1420 including a tablet 1421; snuff 1422; dried and freeze dried fruits and vegetables 1423; and powdered food products for example baking flour 1424 and live powdered drink mixes, live plant powders. Other liposomal encapsulated products 1400 include a cream, gel, and paste liposomal encapsulated product 1430 including a gel 1431; topical cream 1432; cream or gel applied to a tampon 1433 and a paste suppository 1434 of one embodiment.

### Continuous Ultrasonic Cell Disruptor Bath Process:

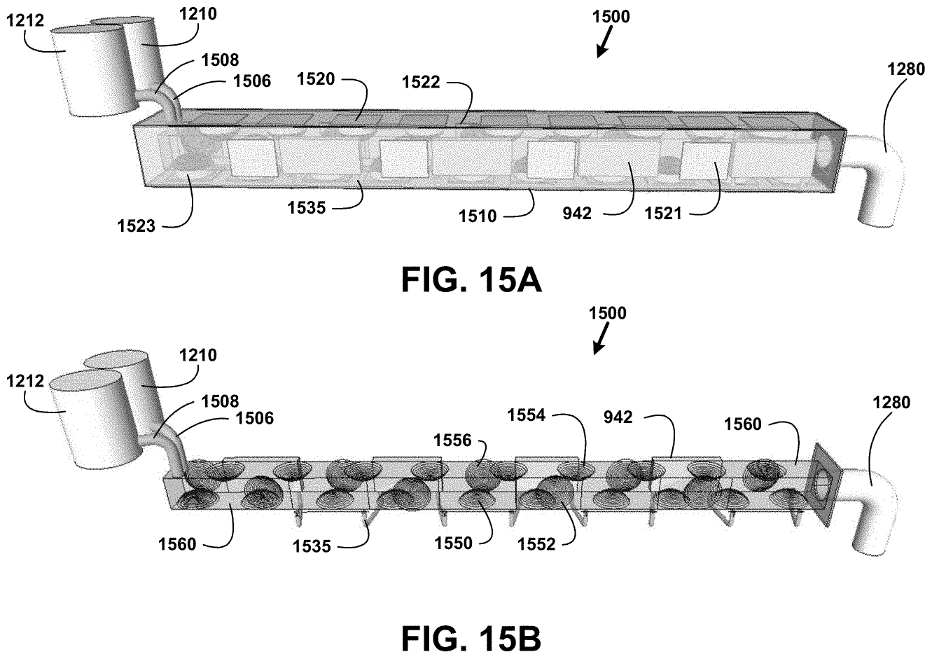

FIG. 15A shows for illustrative purposes only an example of a continuous ultrasonic cell disruptor bath process of one embodiment. FIG. 15A shows a continuous ultrasonic cell disruptor bath apparatus 1500. The continuous ultrasonic cell disruptor bath apparatus 1500 includes a source of cannabinoid from the cannabinoid supply tank 1210 through cannabinoid supply piping 1506 and a source of lipid solution from the lipid solution supply tank 1212 through lipid solution supply piping 1508. A continuous ultrasonic cell disruptor bath apparatus canal housing 1510 shown as translucent for illustration purposes parent covers a continuous ultrasonic cell disruptor bath apparatus canal through which the cannabinoid and lipid solution flow. A plurality of high frequency generators 970 of FIG. 9B are positioned along the continuous ultrasonic cell disruptor bath apparatus canal including at least one high frequency generator top oriented 1520, at least one high frequency generator side 1 oriented 1521, at least one high frequency generator side 2 oriented 1522, and at least one high frequency generator bottom oriented 1523.

The plurality of top, side 1, side 2 and bottom oriented high frequency generators 970 of FIG. 9B create indirect sonication bath high frequency waves through the mixture of cannabinoid and lipid solution flow for processing the sonication of the mixture on a continuous basis and produce the encapsulation process of the mixture creating an encapsulated cannabinoid solution. The encapsulated cannabinoid solution flows out of the continuous ultrasonic cell disruptor bath apparatus canal through encapsulated cannabinoid collection piping 1280.

A plurality of the chilling vessels 942 are positioned on both sides of the continuous ultrasonic cell disruptor bath apparatus canal. The plurality of the chilling vessels 942 extract heat from the sonication caused heating of the cannabinoid and lipid solution as it flows along the canal using a cooling media 944 of FIG. 9B circulated through cooling media circulatory piping 1535 from a cooling apparatus 1224 of FIG. 12 not shown. The sonication process and product process control digital server 1200 of FIG. 12 regulates the volume and flow of the cannabinoid and lipid solution, intensity of the plurality of high frequency generators 944 of FIG. 9B frequency generated, and the temperature and flow of the cooling media 944 of FIG. 9B of one embodiment.

### A Continuous Ultrasonic Cell Disruptor Bath Process High Frequency Pattern:

FIG. 15B shows for illustrative purposes only an example of a continuous ultrasonic cell disruptor bath process high frequency pattern of one embodiment. FIG. 15B shows the continuous ultrasonic cell disruptor bath apparatus 1500 including the cannabinoid supply tank 1210, lipid solution supply tank 1212, cannabinoid supply piping 1506, lipid solution supply piping 1508, cooling media circulatory piping 1535, encapsulated cannabinoid collection piping 1280 and at least one chilling vessel 942. FIG. 9B shows high frequency generator indirect sonication bath high frequency waves 990 of FIG. 9B emanating from the high frequency generators 970 of FIG. 9B and reflecting sonication bath high frequency waves.

FIG. 15B shows high frequency generator indirect sonication bath high frequency waves top oriented 1550, high frequency generator indirect sonication bath high frequency waves side 1 oriented 1552, high frequency generator indirect sonication bath high frequency waves side 2 oriented 1554, and high frequency generator indirect sonication bath high frequency waves bottom oriented 1556 emanating from the plurality of high frequency generators 970 of FIG. 9B positioned along the continuous ultrasonic cell disruptor bath apparatus canal. The mixture of cannabinoid and lipid solution flowing through a continuous ultrasonic cell disruptor bath apparatus canal 1560 is impacted by the indirect sonication bath high frequency waves and reflecting sonication bath high frequency waves continuously causing the cell disruption and encapsulation of the cannabinoids of one embodiment.

### Examples of Liposomal Products Use:

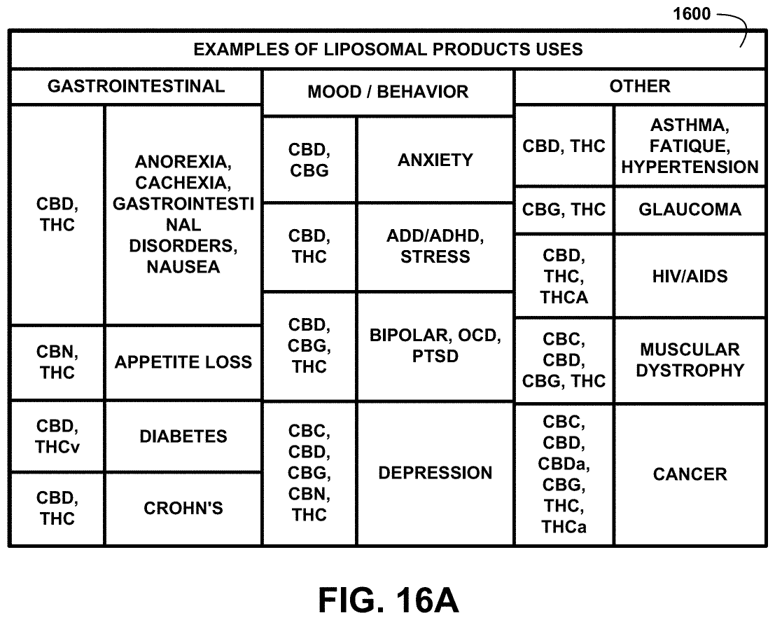

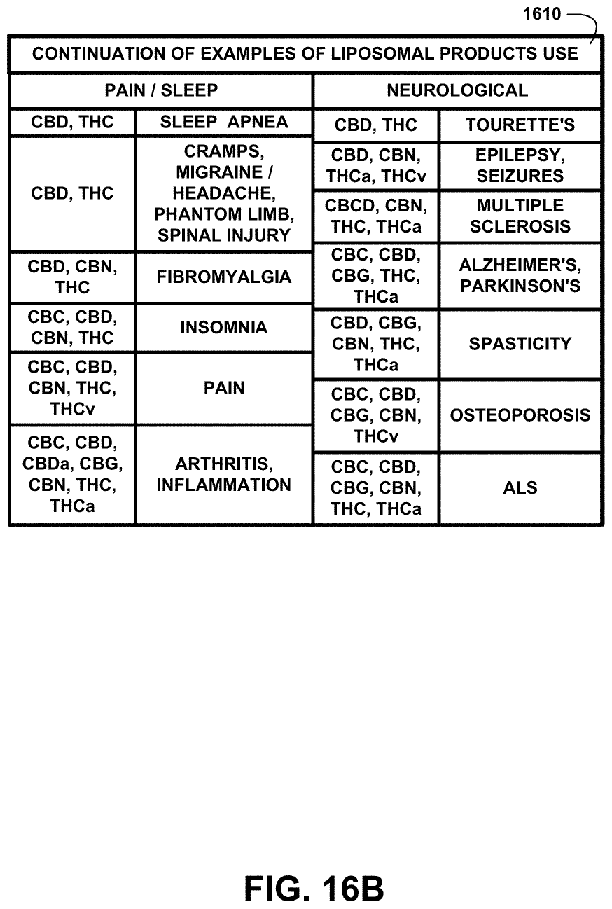

FIG. 16A shows for illustrative purposes only examples of liposomal products use of one embodiment. FIG. 16A shows examples of liposomal products use 1600 including whole cannabis plant derived compounds. The examples of liposomal products use 1600 including whole cannabis plant major cannabinoid compounds and their general use and purpose of one embodiment. The examples of liposomal products use 1600 continue on FIG. 16B.

### Continuation of Examples of Liposomal Products Use:

FIG. 16B shows for illustrative purposes only a continuation of examples of liposomal products use of one embodiment. FIG. 16B shows a continuation of examples of liposomal products use including whole cannabis plant derived compounds for human consumption 1610. The CBD wheel showing whole cannabis plant hemp derived CBD cannabis spectrum 1610 of major cannabinoids and their general use and purpose of one embodiment.

### Examples of Cannabinoid Component Product Uses:

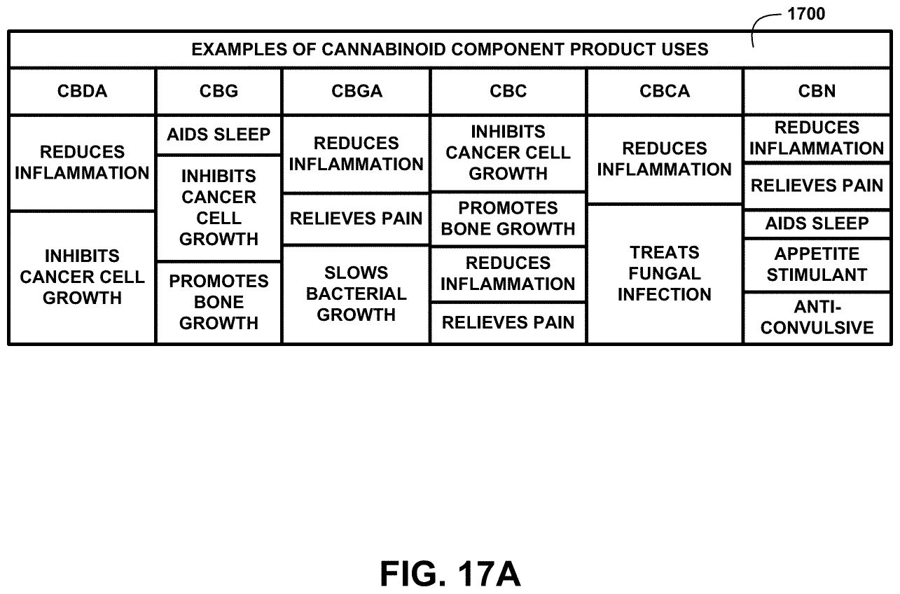

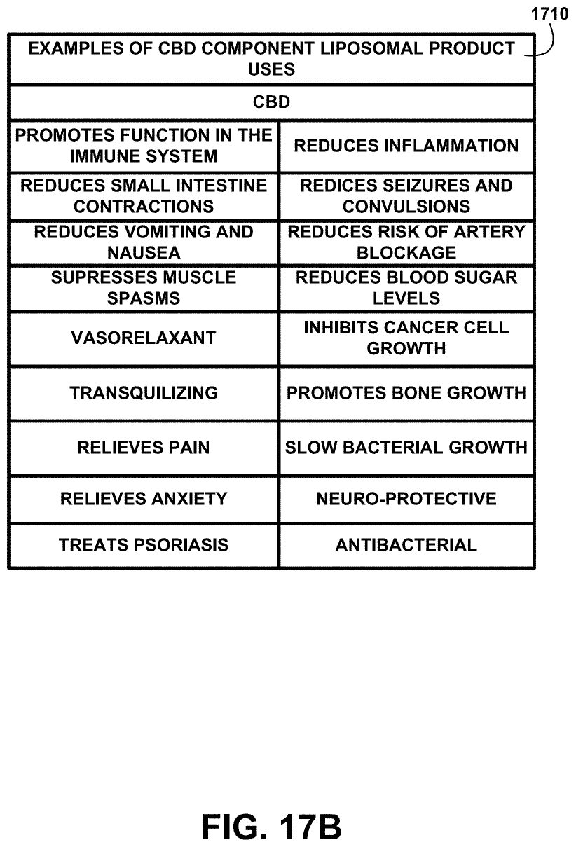

FIG. 17A shows for illustrative purposes only examples of cannabinoid components liposomal product uses of one embodiment. FIG. 17A shows examples of cannabinoid component liposomal product uses 1700 including a listing of major cannabinoid components and the effects of using those major cannabinoid components of one embodiment.

### Examples of CBD Component Liposomal Product Use:

FIG. 17B shows for illustrative purposes only examples of CBD component liposomal product use of one embodiment. FIG. 17B shows examples of CBD component liposomal product use 1710. The examples of CBD component liposomal product use 1710 provide a listing of major CBD component effects of one embodiment.

### Liposomal Encapsulation Process Testing:

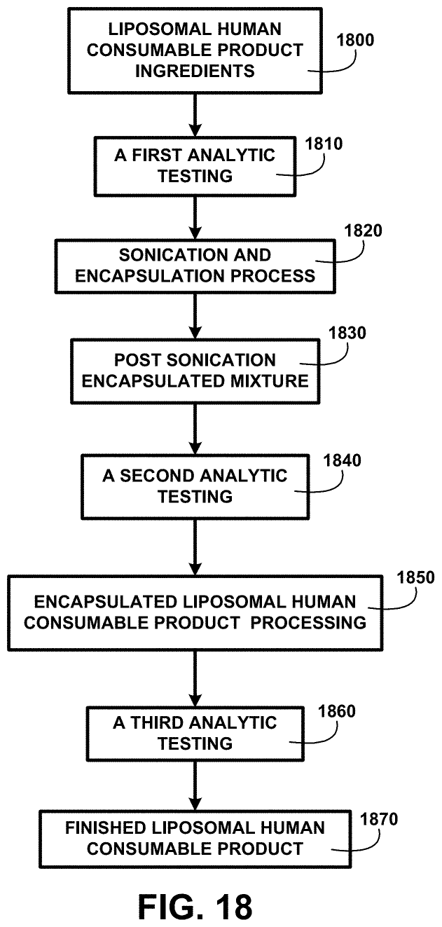

FIG. 18 shows a block diagram of an overview of a liposomal encapsulation process testing of one embodiment. FIG. 18 shows analytical testing of the liposomal human consumable product ingredients 1800 including liposomes, human consumable substances, starch, essential oils and other additive ingredients will be performed at multiple stages in the processing with an approved laboratory. A first analytic testing 1810 will be made prior to mixing the ingredients together. This builds a benchmark analysis for each ingredient. It further provides an opportunity to cull any ingredients that do not meet predetermined quality control standards. Mixing sonication ingredients and performing a sonication and encapsulation process 1820 creates a post sonication encapsulated mixture 1830.

A second analytic testing 1840 is performed for the post sonication encapsulated mixture 1830 to quantify any changes that may have taken place during the sonication and encapsulation processing. A third analytic testing 1860 is performed on each finished liposomal human consumable product 1870. The third analytic testing 1860 establishes the product quality assurance of meeting all predetermined quality control standards. The testing analysis can include calories, pH level, sodium and other mineral content, sugar and glucose content. The analytics used may vary with specific ingredients, for example cannabinoid substances may include percentages of cannabis components including for example THC and CBD potency of one embodiment.

The foregoing has described the principles, embodiments and modes of operation of the embodiments. However, the embodiments should not be construed as being limited to the particular embodiments discussed. The above described embodiments should be regarded as illustrative rather than restrictive, and it should be appreciated that variations may be made in those embodiments by workers skilled in the art without departing from the scope of the present invention as defined by the following claims.

1. A method, comprising:
using a robotic sonication process to encapsulate a human consumable substance within a liposome vesicle;
wherein the robotic sonication processing steps are controlled using a product process control digital server and computer for tracking, regulating and controlling of processing segments;
continuously cooling the liposome vesicle and the encapsulated human consumable substance in a metered and continuous flow using cooling media circulating through piping to and from a cooling heat transfer process apparatus and monitoring temperature readings using a wireless non-contact temperature sensor to a predetermined temperature during the robotic sonication encapsulation process;
adding a starch coating and essential oils after the robotic sonication encapsulation process to the cooled liposome vesicle and the encapsulated human consumable substance to create a mixture;
homogenizing the mixture and processing the homogenized mixture into products in at least one delivery form;
using at least one analytical test for checking predetermined quality control standards of the mixture and products; and
wherein the at least one product delivery form is suitable for human consumption.

2. The method of claim 1, further comprising using an electronic barcode to collect product usage data and transmitting the data to a digital application and wherein using at least one delivery form includes providing cannabinoids for medicinal purposes.

3. (canceled)

4. The method of claim 1, further comprising using a liposome vesicle includes using a liquid lecithin lipid solution including a preferred liquid lecithin using an organic non-GMO Sunflower which includes an elevated hypoallergenic characteristic or a liquid lecithin lipid solution including organic and non-organic soy.

5. The method of claim 1, further comprising using a liposome vesicle includes processing liquid lecithin plus a human consumable material in a one to one ratio plus 7 to 10 extra grams of lecithin depending on the application it is used in for proper cell disruption in a final end product.

6. The method of claim 1, further comprising using a robotic sonication process includes using at least one cell disruptor selected from the group consisting of an ultrasonic cell disruptor probe, an ultrasonic cell disruptor horn cup, and a high frequency generator bath cell disruptor.

7. The method of claim 1, further comprising adding a starch coating includes using maltodextrin for coating the cooled liposome vesicle and the encapsulated human consumable substance mixture for extending shelf life.

8. The method of claim 1, further comprising using at least one product packaging that includes an electronic barcode for gathering data on product usage wherein the electronic barcode is made by embedding at least one digital communication device, a digital processor, a digital memory device and at least one flexible wireless rechargeable battery power supply in an electronic barcode label.

9. The method of claim 1, further comprising gathering data on product usage using a digital application includes an application installed on a product server and a user digital device for recording and displaying product usage.

10. The method of claim 1, further comprising continuously cooling includes using a temperature control cooling apparatus including a chilling vessel that contains a cooling media coupled to a sonication container wherein the cooling media circulates through piping to and from the cooling apparatus to continuously maintain a predetermined temperature during the robotic sonication process.

11-15. (canceled)

16. A method, comprising:
encapsulating a human consumable substance including a cannabis substance within a liposome vesicle using a robotic sonication process;
continuously cooling the liposome vesicle and human consumable substance in a metered and continuous flow using cooling media circulating through piping to and from a cooling apparatus to a predetermined temperature during an encapsulation process;
adding a starch coating and essential oils to the cooled liposome vesicle encapsulated human consumable substance after the robotic sonication encapsulation process;
producing liposome vesicle encapsulated human consumable substance products in at least one delivery form wherein the at least one delivery form is suitable human consumption; and
performing at least one analytical test for checking predetermined quality control standards of the liposome and human consumable substance prior to encapsulation, after encapsulation and upon the encapsulated human consumable substance products.

17. The method of claim 16, further comprising encapsulating a human consumable substance using at least one cell disruptor.

18. The method of claim 16, further comprising performing at least one analytical test including testing a cannabis substance for content percentage of THC, CBD and other cannabis components.

19. The method of claim 16, further comprising encapsulating using a liposome vesicle includes using a liposomal substance including a liquid lecithin including a non-organic and organic non-GMO Sunflower and organic and non-organic soy and starch coating including maltodextrin.

20. The method of claim 16, further comprising encapsulating a human consumable substance includes using a cannabis substance with a liquid lecithin in a one to one ratio plus 7 to 10 extra grams of lecithin based on the encapsulated human consumable substance product application for proper cell disruption in a final end product.
